(19) United States
(12) Patent Application Publication
(10) Pub. No.: US 2024/0306667 A1
(43) Pub. Date: Sep. 19, 2024

(54) PROTEIN RECOVERY FROM PROTEINACEOUS MATERIAL

(71) Applicant: TEAGASC - THE AGRICULTURE AND FOOD DEVELOPMENT AUTHORITY, Carlow (IE)

(72) Inventors: Carlos ALVAREZ GARCIA, Ashtown (IE); Brijesh TIWARI, Ashtown (IE); Dilip RAI, Ashtown (IE); Ahmed KABIR, Ashtown (IE); Shay HANNON, Ashtown (IE)

(73) Assignee: TEAGASC - THE AGRICULTURE AND FOOD DEVELOPMENT AUTHORITY, Carlow (IE)

(21) Appl. No.: 18/682,294

(22) PCT Filed: Aug. 9, 2022
(86) PCT No.: PCT/EP2022/072349

§ 371 Date: Feb. 8, 2024

(30) Foreign Application Priority Data
Aug. 10, 2021 (EP) 21190703.5

(51) Int. Cl. A23J 1/14; A23J 1/00; A23J 3/14; C07K 1/14

(52) U.S. Cl. CPC A23J 1/14; A23J 1/005; A23J 3/14; C07K 1/145

(57) ABSTRACT

Provided is a method of protein recovery from a plant by-product. The method comprises isoelectric solubilisation of the plant by-product with an alkali solution to provide a first solubilised protein fraction, separating the first solubilised protein fraction from unsolubilised plant matter; and flocculating the first solubilised protein fraction with an amount of a flocculating agent to provide a protein product.

### FIELD OF THE INVENTION

The invention relates to methods for recovering protein from proteinaceous plant material. In particular, the invention relates to method for recovering protein from rapeseed plant material.

### BACKGROUND TO THE INVENTION

There is an increasing emphasis on the nutritional importance of a plant-based diet and on plant proteins as alternatives to animal proteins. Oilseeds of the Brassica family, such as rapeseed, have the potential to be used in food, but are rarely used as a food component (Chmielewska et al., Critical reviews in food science and nutrition, 2000). The processing of rapeseed seeds for oil production provides an oilseed cake co-product, also known as rapeseed press cake (RPC). RPC raw material is composed mainly of fibre (50-60%), protein (30%), oil (3-5%) and moisture (5-8%) (Ghazani, S. M., et al., 2014, European Journal of Lipid Science and Technology, 116(4), 380-387; Matthäus, B., et al., (2003). Food/Nahrung, 47(6), 413-419). In its original composition, several undesirable compounds can be found, which limit the further application of this product as a protein-rich ingredient. Such compounds possess anti-nutrient properties and a strong bitter taste. These compounds need to be removed if the RPC is to have a beneficial use in food or beverage products. Thus, RPC requires further processing in order to be employed as a protein-rich ingredient in such products.

The current state of the art provides several methods for protein recovery from proteinaceous material. These include enzymatic extraction, water extraction, isoelectric solubilisation precipitation methodology (ISP) or ISP assisted by US (ultrasound). Several groups have reported methods to extract proteins from plant sources.

Teh SS, et al., (Food Measure 8, 92-104 (2014) investigated the effect of the defatting process, acid and alkali extraction, on the physiochemical and functional properties of hemp, flax and rapeseed seed cake protein isolates. Ghodsvali A. et al., (Food Research International, Volume 38, Issue 2, 2005, Pages 223-231) disclose conditions for the extraction and precipitation of proteins from Iranian rapeseed. The method involved a membrane-based filtration process which consisted of extraction of hexane-defatted rapeseed means at pH 9.5-12 and precipitation at pH values between 3.5 and 7.5 to recover a precipitated protein isolate. Rodrigues et al., (J Sci Food Agric, 2017 June; 97(8): 2641-2646) discloses a method of protein extraction from rapeseed meal using a pre-treatment with phytase in order to remove anti-nutrient (“metal-chelating”) phytic acid. This process involves defatting the raw material using petroleum either. Extraction is performed at high temperatures (55° C. to 75° C.). This method provided a product with phytic acid levels of ˜1 g kg⁻¹. The same group also investigated using enzymatic hydrolysis of carbohydrates and subsequent removal to increase the protein content of rapeseed meal (Rodrigues et al., 2014, Bioresources, 9(2), 2010-2025) and thus its nutritional value. It does not involve ISP of protein.

Gillberg, L., et al., (Journal of Food Science, 1976, 41(5), 1070-1075) discloses preparation of rapeseed protein isolates in the presence of polyacids. The method uses a defatted raw material using an organic solvent. Extraction was carried out at pH 11.1.

There are currently two main methods used in the industry to purify and extract rapeseed protein products. EP3520624 discloses the use of organic solvents (ethanol) followed by an enzymatic treatment to reduce the content in phytates; completed by a high temperature treatment. As a result, a final product with ˜40% protein content is obtained. No yield is reported in the invention.

WO2017102535A1 describes a process in which high ionic solutions (NaCl at high molarity) are employed to solubilize target proteins. After this, salt is diluted by adding a considerable amount of water and the proteins can be then recovered by centrifugation or filtration. By this process a final product with ˜90% proteins content is obtained, with a protein recovery yield less than 20%.

O Paredes-Lopez et al (Chickpea protein isolates: physiochemical, functional, and nutritional characterisation, Journal of Food Science, vol. 56, no. 3, 1 May 1991) discloses a method of isolating proteins from chickpea flour by micellization and isoelectric precipitation.

Daniela Von Der Haar et al (Rapeseed proteins-production methods and possible application ranges, OCL, vol. 21, no. 1, 1 Jan. 2014) compares two strategies for producing functional ingredients from rapeseed. One process is based on the use of alkaline extraction using a starting material of defatted rapeseed meal. This method requires the use of organic solvents to remove the fat from the starting material. Allowing to reach a final protein content in the extract of 80% to 90%; nevertheless, no mention about protein recovery yields is provided. Taskaya Lafit et al (Compositional Characteristics of Materials Recovered from Whole Gutted Silver Carp using isoelectronic solubilisation/precipitation, Journal of Agriculture and Food Chemistry, vol. 57, no. 10, 27 May 2009) describes isoelectric solubilisation at acidic and basic pH with whole carp and the use of the produced product in human food and animal feeds.

The demand for plant protein, especially rapeseed protein, is increasing globally and, therefore, there is a need to provide a product that is rich in protein and low in undesired components. The current invention serves to provide this while overcoming the problems associated with the prior art methods. As a currently underused co-product, the current invention allows RPC to be revalorised, recovered, and used as an emerging source of edible protein with high nutritive value.

### SUMMARY OF THE INVENTION

The current invention uses the principles of ISP with flocculation to separate proteins from undesirable compounds, such as phytic acid, erucic acid, glucosinolates and fibre, in proteinaceous plant material to obtain a final product with a high protein content ranging from 62% to 66%, and a desirable protein recovery yield of about 50%. Advantageously, the final protein content and yield obtained using the method of the invention are significantly and surprisingly better than those obtained using prior art methods, including those processes available in the market.

The final product is rich in protein, low in bitter taste and anti-nutrient compounds. Moreover, surprisingly, the properties and characteristics of the final product differ significantly when compared to existing rapeseed extracted protein products (Table 1). Such characteristics include the protein content of the final product (62-66%), the presence of beneficial fatty acids (mono and polyunsaturated fatty acids) and a 5-6% of dietary fibre. Finally, the amino acid profile of the final product is not modified when compared to the raw material, which indicates that the nutritional profile is not affected by the current method.

A further advantage is that the current method avoids the use of organic solvents as it does not use a defatting step. The method also employs regular standard equipment already available in the food industry sector.

Broadly, an aspect of the current invention provides a method of protein recovery from a plant by-product, the method comprising the steps of;
isoelectric solubilisation of the plant by-product with an alkali solution or acid solution to provide a first solubilised protein fraction,
separating the first solubilised protein fraction from unsolubilised plant matter;
flocculating the solubilised protein fraction with an amount of a flocculating agent to provide a protein product.

In an embodiment, the isoelectric solubilisation is with an alkali solution.

Typically, the method further comprises separating the insoluble flocculated protein product from soluble matter, or soluble faction. The soluble matter is in the liquid phase.

Preferably, the flocculating agent is one or more agents selected from the group comprising sodium hexametaphosphate, alginate, carboxymethylcellulose (CMC), polyacrylic acid, tannic acid. Preferably, the flocculating agent is CMC.

Preferably, flocculating the solubilised protein fraction is carried out at an acidic pH, preferably pH of between 4.5 and 5.5. Preferably a pH of 5.

Typically, the method comprises neutralising, or adjusting, the pH of the separated first solubilised protein to a pH of from 4.5 to 5.5, preferably a pH of 5, prior to, or simultaneously with, flocculating the solubilised protein fraction. Preferably, an amount of an acidic solution is added to the solubilised protein fraction to reach the required pH in an embodiment in which the isoelectric solubilisation is carried out with an alkali solution. Typically, an amount of an alkali solution is added to the solubilised protein fraction to reach the required pH in an embodiment in which the isoelectric solubilisation is carried out with an acid solution. Once added, this provides a protein suspension.

In an embodiment, the plant by-product is one produced during oil production utilising the plant. It may be from the seed, or seeds, of the plant.

Preferably, the plant is one from the genus Brassica.

Preferably, the plant is a rapeseed plant, and the plant by-product is a rapeseed press cake (RPC) or pellets.

Typically, the plant is a rapeseed plant.

In an embodiment, the protein product is subsequently freeze dried and/or milled.

In an embodiment, separation of the current method may be by any suitable means. Preferably, separation is by centrifugation. Other means of separation may be employed, and these means are known in the art. The means includes but is not limited to filtration and membrane separation (microfiltration or ultrafiltration).

In an embodiment, separation is carried out at a temperature of less than 30° C., preferably 25° C. or less, typically about 25° C.

Preferably, isoelectric solubilisation of the plant by-product with an alkali solution is carried out at a pH from 11.2 to 12.5, preferably a pH of 12.

The end product of protein recovery may be a powder, or a paste, or a concentrated protein solution.

Preferably, the concentration of the flocculating agent to be added is about 0.5 to 1.5% (w/v) preferably 1% w/v, with respect of the protein solution obtained after the alkaline extraction step. Flocculating agent solution is added to the separated solubilised protein fraction at a ratio of 1:10 (v/v), giving a final CMC concentration of 0.1% w/v.

Preferably, the step of flocculation is under agitation. For example, in a stirred reactor or similar.

In an embodiment, the method optionally comprises hydrolysing any polysaccharides in the first solubilised protein fraction prior to flocculating the protein fraction. This step may be carried out by adding an amount of a suitable enzyme. The enzyme may be viscozyme. Preferably, the pH of the first solubilised protein product is adjusted to a pH of 3.5 prior to hydrolysis.

In one embodiment, the method comprises a step of isoelectric solubilisation of the unsolubilised plant matter to provide a second solubilised protein fraction. The method may further comprise a step of separating the second solubilised protein fraction from the unsolubilised plant matter in this embodiment. This second isoelectric solubilisation step may be carried out in an acidic solution or an alkali solution. Thus, the first isoelectric solubilisation step may be carried out in acid solution and the second isoelectric solubilisation step carried out in alkali solution, or vice-versa.

The first solubilisation step provides precipitate of unsolubilised matter and a supernatant containing a first solubilised protein fraction and the second solubilisation step is carried out on the precipitate of the first step and solubilises the proteins that are not solubilised in the first step.

In this embodiment, the first and second solubilised protein fractions are then combined prior to flocculating the combined protein fraction with a suitable flocculating agent, such as CMC. In this method of sequential extraction, the supernatants, i.e., the first solubilised protein fraction and the second solubilised protein fraction, can be mixed to bring the pH to a pH from 4.5 to 5.5, preferably a pH of 5. The two fractions are combined in such a way that the pH is between 4.5 and 5.5, or the pH may be adjusted to a pH of from 4.5 to 5.5 using an alkali solution or acidic solution.

In an embodiment, the plant by-product is treated with ultrasound during one, or both, of the isoelectric solubilisation steps.

In an embodiment, the isoelectric solubilisation step(s) is carried out under agitation. For example, in a stirred reactor or similar.

In an embodiment, when the method is one of sequential ISP, the first isoelectric solubilisation step may be carried out on a first batch of plant by-product and the second isoelectric solubilisation step may be carried out on unsolubilised plant product from a second batch of plant by-product and wherein the two steps are carried out simultaneously.

In an embodiment, the plant by-product is size reduced prior to the isoelectric solubilisation step, typically to a powder. Various size reduction steps are envisaged including mincing, grinding, homogenisation, depending on the nature of the plant by-product.

Typically, the method may optionally comprise an initial step of producing an oil seed cake, such as RPC, from a plant seed. Preferably, the initial step comprises cold pressing a plant seed to extract oil to provide an oilseed cake. This may then be dried into a pellet.

In an embodiment, the alkaline extraction step takes place at room temperature, e.g., from 20° C. to 25° C., preferably 25° C. In an embodiment, the entire method takes place at room temperature, e.g., from 20° C. to 25° C., preferably 25° C.

In an embodiment, the alkaline extraction step takes place at from 35° C. to 50° C., preferably at 45° C. In this embodiment, the method may comprise a step of heating the plant-by-product prior to the alkaline extraction step and maintaining this temperature for the duration of the alkaline extraction step. This embodiment provides an even lower level of phytochemicals in the final product.

In an embodiment, the solution is allowed to cool, e.g., until it reaches room temperature, after the alkaline extraction step is carried out and before the flocculation step.

In an embodiment, the flocculation step takes place at from 35° C. to 50° C., preferably at 45° C. Alternatively, the flocculation step takes place at room temperature, or another temperature suitable for this step to be carried out to complete this action as required. A further aspect of the invention provides a plant protein product, or plant protein concentrate, obtained from the method of the invention.

A further aspect of the invention provides a plant protein product, or a plant protein concentrate, comprising from 60% to 70% protein content, from 1% to 10% fibre, from 20% to 25% lipids, such as fatty acids, and from 35% to 40% essential amino acids.

Preferably, the plant protein product or concentrate comprises from 62% to 66% protein content, from 5% to 6% fibre, from 20% to 22% lipids, such as fatty acids, and 37% to 39% essential amino acids.

The essential amino acids may be one or more of those listed in Table 7. The essential amino acids may be all of the amino acids listed in Table 7.

The invention also provides a food or beverage product comprising the plant protein product or concentrate of the invention.

The invention also provides a food ingredient comprising the plant protein product of the invention.

The food product may be a protein extender, a meat replacement, or a meat analogue. Typically, the plant protein product or concentrate is a powder.

### DEFINITIONS AND GENERAL PREFERENCES

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g., a feature, element, characteristic, property, method/process step or limitation) or group of integers (e.g., features, element, characteristics, properties, method/process steps or limitations) but not the exclusion of any other integer or group of integers. Thus, as used herein the term “comprising” is inclusive or open-ended and does not exclude additional, unrecited integers or method/process steps.

“Rapeseed press cake (RPC)” is a by-product, or co-product, of the processing of rapeseed seeds for edible oil production. Generally, RPC raw material is composed mainly of fibre (50-60%), protein (30%), oil (3-5%) and moisture (5-8%). It can be provided in the form of wet paste (or cake) after oil production, or a pellet. The pellet is when the cake is dried to have a lower moisture content.

“Rapeseed” (Brassica napus subsp. napus) is a member of the family Brassicaceae. It is cultivated mainly for its oil-rich seed. “Rapeseed” is a group of rapeseed cultivars which are bred to have low levels of erucic acid. “Rapeseed oil” is a vegetable oil derived the rapeseed variety of rapeseed.

The genus “Brassica” is a genus of plants in the cabbage and mustard family (Brassicaceae). Species of Brassica include but are not limited to Brassica napus, Brassicae juncea and Brassica rapa. Brassciae napus includes rapeseed, rutabaga and Siberian kale.

The term “isoelectric precipitation/solubilisation” or ISP means exposing plant matter to an acid or alkali solution for a period of time sufficient to allow solubilisation of at least a part of the protein in the plant matter. Subsequently, the pH of the solution with the solubilised proteins is shifted to the isoelectric points of the proteins in solution. Generally, each isoelectric solubilisation step is carried out for at least 5, 10, 15 or 20 minutes.

The term “sequential isoelectric solubilisation/precipitation” should be understood to mean a process comprising a first isoelectric solubilisation step, which produces a precipitate of unsolubilised material, and a second isoelectric solubilisation/precipitation step carried out on the precipitate produced in the first isoelectric solubilisation step. The first step may be alkali solubilisation and the second step acid solubilisation, or the first step may be acid solubilisation and the second step acid solubilisation.

The term “plant by-product” as used herein refers to a proteinaceous material obtained from plants, such as a rapeseed plant, suitable for processing for recovery of protein. Typically, the by-product is obtained from a plant processing method, such as plant oil production or similar, e.g., rapeseed oil production. It may be or sourced from any suitable matter derived from a plant, e.g., one or more of roots, stems, leaves, flowers, fruits and seeds.

The term “acid solution” as used herein means a solution having a pH of less than 5, and generally having a pH of 1-4, preferably 2-3. It will generally be a strong acid solution. Examples of acid solutions include several molarities of mineral or organic acids. Typically, the acid solution has a concentration of 0.1-0.4 moles/litre, generally the concentration required to reach the desired value, typically 2-3. In one embodiment, the acid is selected from hydrochloric acid, phosphoric acid, citric acid, sulphuric acid or acetic acid. The term “alkali solution” means a solution having a pH of greater than 8, and generally having a pH of 9-12, preferably 11-12, most preferred 12. It will generally be a solution of a caustic compound. Typically, the alkali solution has a concentration of 0.1-0.4 moles/litre. Typically, the alkali is selected from calcium hydroxide, sodium hydroxide, potassium hydroxide or ammonium hydroxide. The concentration of the alkali solution used in this method may be from 1M to 6M, 2M, 3M, 4M, or 5M.

The term “solubilised protein fraction” as used herein means a fraction containing solubilised protein and substantially free of unsolubilised matter. Generally, the solubilised protein fraction is separated from the unsolubilised material using well known separation techniques, such as filtration, decantation, or membrane separation. Examples of membrane separation include microfiltration and ultrafiltration.

The term “recovery of protein” is the isolation or extraction of protein from a sample. It can include concentration of the protein, flocculating the protein, drying of the protein fraction, and/or isolation of protein by for example precipitation. In one embodiment, protein recovery is achieved by means of precipitation, such as isoelectric solubilisation/precipitation. Drying can be achieved by evaporation, spray drying, lyophilisation, or other means including nanofiltration.

The term “stirred reactor” is composed of a tank or container and a mixer such as a stirrer, a turbine wing or a propeller.

The term “size-reduced” means treating the plant material to reduce the size of the material. Examples of size-reduction processes include mincing, homogenisation and milling. Generally, when the material is homogenised, it is homogenised in an acid or alkali solution.

The term “flocculation” when used herein refers to a process by which proteins form or cause to form small clumps or aggregates, by means of the addition of a “flocculating agent”. In this process, the proteins come out of suspension to sediment. A “flocculating agent” is an agent that actions this process.

The term “flocculated protein product”, or “aggregated protein product” refers to the complex formed by the flocculating agent and proteins (previously solubilised and then placed at their isoelectric pH) stabilised by electrostatic interactions.

“Carboxymethyl cellulose (CMC)”is a cellulose derivative with carboxymethyl groups (—CH₂—COOH) bound to some of the hydroxyl groups of the glucopyranose monomers that make up the cellulose backbone.

### BRIEF DESCRIPTION OF THE FIGURES

The current invention will now be described with reference to the following Figures in which;

FIG. 1: Process 1 involving alkaline extraction in combination with carboxymethyl cellulose (CMC) treatment.

FIG. 2: Process 2 involving alkaline extraction in combination with CMC and viscozyme L® treatment.

FIG. 3: Process 3 involving sequential alkaline an acid extraction in combination with CMC.

FIG. 4 (A) and (B): Emulsion activity of the product of Process 1 and 2 (A) and emulsion stability index of the product of Process 1 and 2 (B).

FIG. 5 (A) and (B): Foaming capacity of the product of Process 1 and 2 (A) and foaming stability of the product of Process 1 and 2 (B).

FIG. 6: Rapeseed extraction process of an embodiment of the invention.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Plant by-products, especially RPC, are currently underused co-products but have the potential to be used as an edible source of protein with high nutritive value. However, there is a need to provide a method of extracting or recovering these protein products which removes unwanted compounds but also provides a final product with a high protein content and protein yield. The current invention serves to provide such a method.

The current method provides a method of protein recovery, or extraction, from a plant-by product using isoelectric solubilisation synergistically with flocculation. The effect of acidic conditions promoting protein precipitation are surprisingly enhanced when combined with the flocculant.

When compared to other processes specific for plant protein extraction, particularly rapeseed plant protein extraction, the current invention has, but not limited to, the following advantages:
The balance between recovery yield (50%) and final protein content (63-65%) is significantly better when compared to other prior art methods currently used as standard in the industry.
The current method does not require the use of organic solvents.
The current method does not involve any heat or high temperature process.
Water consumption of the current method is significantly lower when compared to the process based on high ionic extraction buffers.
The current method can easily be adopted by any food processor since regular equipment is employed.
The extraction process can be completed within two hours, exclusive of drying time and packaging.

It will be appreciated that the parameters of the process (pH, solvent/sample ratio, extraction time and temperature) can be modified according to the plant protein source.

The final product is rich in protein, low in bitter taste and anti-nutrient compounds. Moreover, surprisingly, the properties and characteristics of the final product differ significantly when compared to existing rapeseed extracted protein products (Table 1).

TABLE 1
Properties of the product of the method of the invention compared with
protein products of the prior art. Isolexx ® and Vitalexx ® products were
generated using the method of WO2017102535. The products are based
on protein extracted from rapeseed.

| | % Protein content | | | % essential |
|---|---|---|---|---|
| Properties | in final product | % Fibre | % Lipids | amino acids |
| Our invention | 62-66 | 5-6 | 20-22 | 37-39 |
| Avena product | 33-43 | 33-43 | 14-22 | NR |
| | | | | (not |
| | | | | reported) |
| Isolexx ® product | >90 | <0.1 | <2 | 37 |
| Vitalexx ® product | >80 | <10 | <0.5 | 39 |

The process of the invention is based on a completely different biochemical principle compared with existing technologies.

The current method does not comprise any defatting step, compared with prior art methods which use a defatting step on the starting product with organic solvents. In contrast, the starting product of the current method is not defatted, e.g., a non-defatted press cake.

The starting plant by-product may be mixed with a buffer prior to extraction in order to rehydrate the starting material. This may be any suitable buffer, for example, tap water, demineralised water or distil water. The preferable ratio may range from 1:6 to 1:20 w/v, although the preferable ratio may be of 1:10 w/v.

In an embodiment, all steps of the method are carried out at a temperature of 30° C. or less, preferably, 25° C. or less, or between 15° C. and 25° C. As a result, no energy is required for heating up the solutions as the method can be carried out at room temperature.

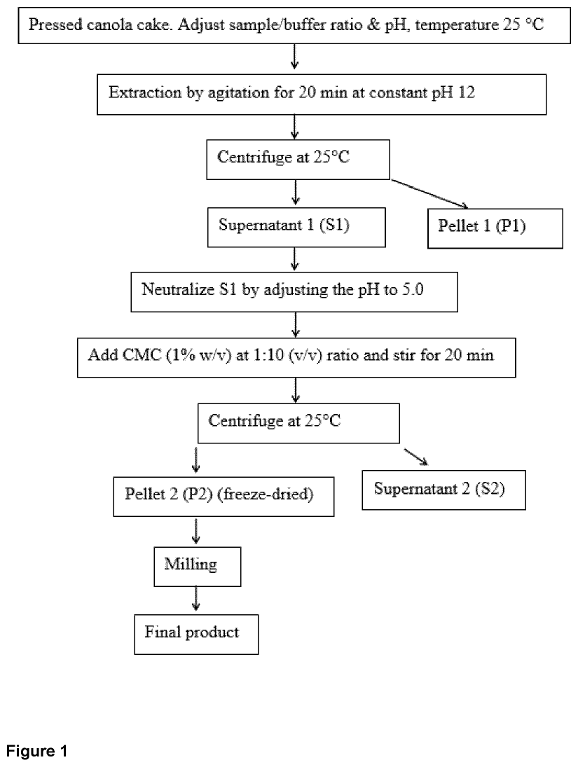

The process flow chart of an embodiment of the invention is illustrated in FIG. 1 and an illustrated example is provided in Example 1.

Briefly, the plant raw material, in this case RPC raw material, is mixed with water and the pH adjusted to a final value of 12 with a volume of NaOH. In this example, the pH is 12. However, the pH in the method of the invention may be one between 11.2 and 12, such as 11.3, 11.4, 11.5, 11.6, 11.7, 11.8, 11.9.

After the solubilization step an aqueous phase rich in soluble proteins (S1) is obtained after separation, along with a non-soluble fraction composed of plant material and remaining insoluble proteins (P1). Separation in this instance is by centrifugation. The pH can then be dropped to a desired value, e.g., neutralised by adjusting to pH 5, by adding an amount of a suitable acid. The pH may be one that promotes precipitation. This may be HCL ranging from 2 to 6M concentration, e.g., 3, 4, or 5M. An amount of a flocculating agent is then added. In this instance, CMC is added. This provides an aggregated or flocculated protein product. Optionally, the protein product can then be separated from solubilised matter in liquid phase by centrifugation. A second precipitate (P2) is obtained together with a supernatant (S2) by separation using centrifugation. The pellet, P2, is rich in protein. The pellet can be freeze dried and optionally milled, to produce a final protein product. The supernatant is rich in minerals, some soluble carbohydrates, and fibres. It is non-flocculated soluble matter.

Some small amounts of protein may remain soluble in the final supernatant after protein centrifugation; such proteins can easily be recovered and desalted by membrane filtration.

In the method of the invention, notably the flocculating agent may be one or more agents selected from the group comprising sodium hexametaphosphate, Alginate, carboxymethylcellulose (CMC), polyacrylic acid, tannic acid. It will be appreciated that any flocculating agent that is capable or suitable for the necessary action may be used. A suitable amount of the flocculating agent may be used, i.e., an amount capable of flocculating the protein. It will be appreciated that determining a suitable amount is part of the knowledge of the person skilled in the art. Preferably, the flocculating agent is CMC.

Typically, the concentration of the flocculating agent to be added is about 0.5 to 1.5% (w/v) preferably 1% w/v. Typically, flocculating agent solution is added to the separated solubilised protein fraction at a ratio of 1:10 (v/v), giving a final CMC concentration of 0.1% (w/v). The ration is about 1:5 to 1:20, preferably 1:10 (v/v).

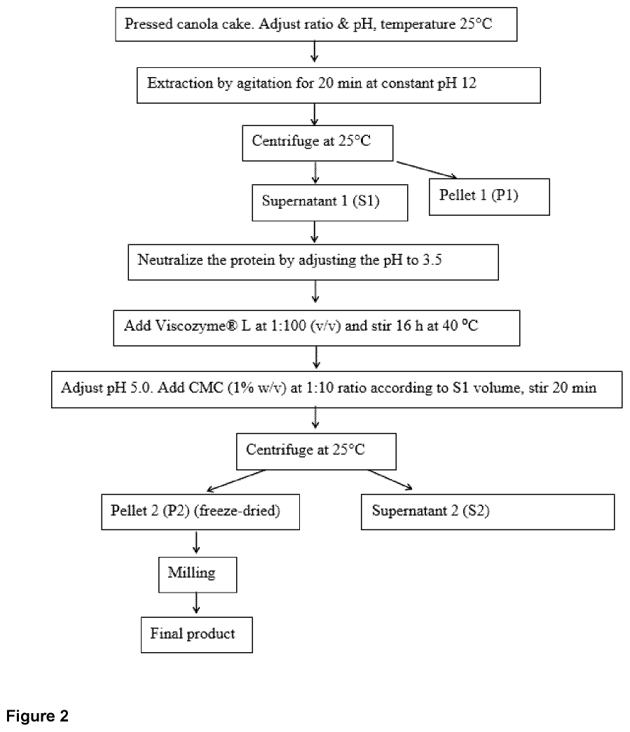

The process flow chart of an embodiment of the invention is illustrated in FIG. 2 and an illustrated example is provided in Example 2. The steps are the same as those of process up until the point that S1 is obtained and separated from P1. The pH is dropped to the desired value and an amount of viscozyme is added to S1. After this point, the steps of the method are the same as those of process 1.

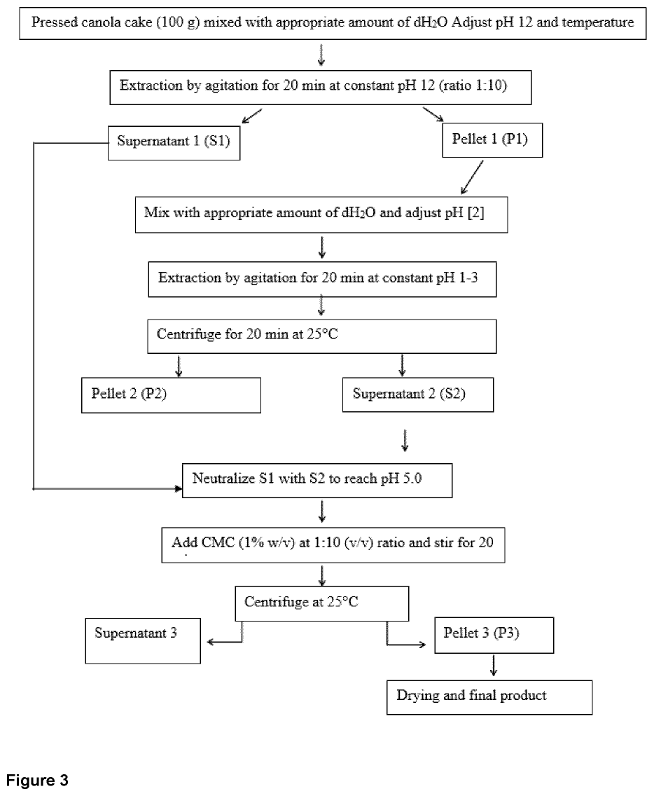

The process flow chart of an embodiment of the invention is illustrated in FIG. 3. This is sequential extraction. The steps are the same as those of process up until the point that S1 is obtained and separated from P1. The method then comprises a second step of isoelectric solubilisation of the unsolubilised plant matter (P1) in an acid solution to provide a second solubilised protein fraction. The second solubilised protein fraction is from the unsolubilised plant matter in this embodiment to provide S2. This second isoelectric solubilisation step may be carried out in an acid solution or buffer.

In this embodiment, the first and second solubilised protein fractions are then combined prior to addition of the flocculating agent, in this instance CMC. Typically, the volume and concentration of the acid buffer employed for the second extraction is such that one combined with the alkaline solution the pH is 5.

The first solubilisation step provides precipitate of unsolubilised matter and a supernatant containing a first solubilised protein fraction and the second solubilisation step is carried out on the precipitate of the first step and solubilises the proteins that are not solubilised in the first step.

In one embodiment, both supernatants rich in soluble proteins are mixed in the right proportion to get a final blend with a pH value that promotes precipitation of the solubilised protein, for example a pH of 5.5.

Thus, in one embodiment, the protein from the first and second solubilised protein fractions are recovered by combining the first and second solubilised protein fractions in a proportion to effect precipitation of the protein. The supernatants may be combined in amounts to effect precipitation. The supernatants may be combined proportionally to provide a weakly acidic solubilised protein fraction having a pH of 5 to 6, preferably 5.5.

By adding this extra step of acidic extraction, an extra 1% in protein recovery yield could be achieved; with no further increase in final protein content.

TABLE 2
Results provided extracting or recoverin proteins at acid pH
using the pellet obtained after alkaline extraction as raw material.

| | | | Pellet | | | | |
|---|---|---|---|---|---|---|---|
| | | | (g) | | Freeze | | Protein |
| | | Ratio | 20% | | dried | Protein | Re- |
| | | (pellet/ | solid | volume | sample | content | covered |
| Trials | pH | water) | content | (mL) | (g) | (%) | (g) |
| 1 | 2 | 0.115 | 100 | 173 | 0.48 | 10.99 | 0.05 |
| 2 | 3 | 0.15 | 100 | 133 | 0.49 | 7.59 | 0.04 |
| 3 | 3.41 | 0.115 | 100 | 173 | 0.45 | 15.92 | 0.07 |
| 4 | 2 | 0.115 | 100 | 173 | 0.52 | 12.54 | 0.07 |
| 5 | 3 | 0.08 | 100 | 250 | 0.86 | 14.27 | 0.12 |
| 6 | 2 | 0.16 | 100 | 121 | 0.70 | 9.01 | 0.06 |
| 7 | 1 | 0.15 | 100 | 133 | 0.60 | 10.10 | 0.06 |
| 8 | 1 | 0.08 | 100 | 250 | 0.53 | 19.31 | 0.10 |
| 9 | 2 | 0.065 | 100 | 305 | 0.50 | 12.22 | 0.06 |
| 10 | 0.59 | 0.115 | 100 | 173 | 1.00 | 9.94 | 0.10 |
| 11 | 2 | 0.115 | 100 | 173 | 1.19 | 9.15 | 0.11 |

This method also comprises optional recovery of the protein from the first and/or second solubilised protein fraction.

Importantly, the method of the invention is a process that can be easily scaled, since just larger stirred reactors, and industrial scale decanters or separators (currently used in food industry) are needed to complete the process.

This method provides an opportunity for the plant processing industry to increase profitability by implementing an economical and efficient process capable of generating protein based added-value products.

Notably, the invention provides a plant protein product, or a plant protein concentrate, comprising from 60% to 70% protein content, from 1% to 10% fibre, from 20% to 25% lipids, such as fatty acids, and from 35% to 40% essential amino acids.

The protein content may be 60%, 61%, 62%, 63%, 64%, 65%, 66%, 67%, 68%, 69%, or 70% or greater. It may be from 60% to 65%, or from 60% to 70%. It may be at least 60% or 65%. The fibre content may be 1%, 2%, 3%, 4%, 5%, 6%, 7%, 8%, 9% or 10%. It from be from 1% to 10%, or 2% to 8%, or 3% to 5%. The lipid content may be 20%, 21%, 22%, 23%, 24% or 25%. It may be from 20% to 25%.

The protein product may contain 35%, 36%, 37% 38%, 39% or 40% essential amino acids. It may be at least 35%. It may be from 35% to 40%.

It will be appreciated that the product may have any combination of the above disclosed protein, fibre, lipid and amino acids. Of note, an aspect of the invention provides a plant protein product that comprises from 62% to 66% protein content, from 5% to 6% fibre, from 20% to 22% lipids, and 37% to 39% essential amino acids.

The final product obtained also has distinct properties, being richer in mono-unsaturated fatty acids (MUFAs) and poly-unsaturated fatty acids (PUFAs) and dietary fibre, and vegan and vegetarian consumers will accept it.

The essential amino acids are one or more of those listed in Table 7.

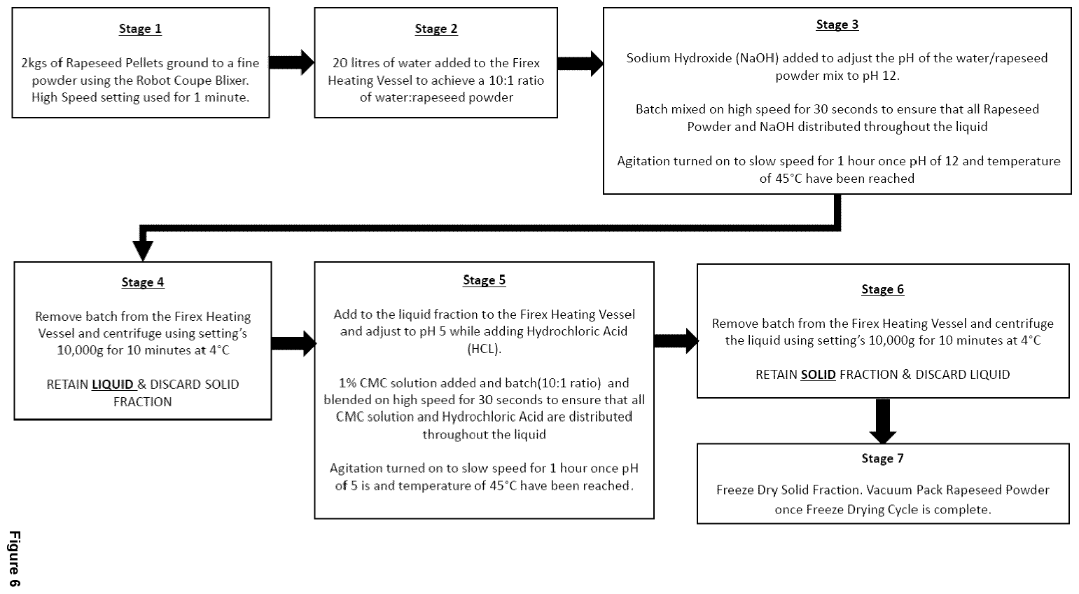

The process flow chart of an embodiment of the invention is illustrated in FIG. 6. In this embodiment, the step of alkaline extraction and the step of flocculation at acidic pH are carried out at 45° C. Briefly, in this method stage 1 comprises grinding a rapeseed pellet to a powder to provide a starting product. In stage 2, 20L of water is added to the powder product to achieve a 10:1 ratio of water: powder. Stage 3 comprises isoelectric solubilisation with an alkaline solution. In this example, NaOH is then added to provide a pH of 12. Agitation takes place at a pH of 12 and a temperature of 45° C. Separation in this instance is by centrifugation. The mixture is then centrifuged at 10000 g for 10 minutes at a temperature of 4° C. The liquid fraction is retained, and the pH adjusted to 5 in this example. The flocculation step involves addition of 1% CMC. Agitation takes place at a pH of 5 and a temperature of 45° C. Separation in this instance is by centrifugation. The mixture is then centrifuged at 10000 g for 10 minutes at a temperature of 4° C. The solid fraction is retained. In this instance it is freeze dried and vacuum packed. This embodiment provides an even lower level of phytochemicals in the final product (FIG. 7).

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### EXAMPLE 1

In the process illustrated in FIG. 1, RPC is mixed with tap water to a ratio 1:10 (w/v), (4 kg in 36 litres) and the pH is adjusted to a final value of 12 by means of adding an alkali solution (6M NaOH, around 600 mL). Although 25° C. is noted in FIG. 1, but temperature does not need to be controlled as the room temperature is sufficient. Values of pH were controlled after thoroughly stirring the mixture using a calibrated pH probe.

Centrifugation step was conducted using two devices a) large capacity centrifuge (batch process), where 10,000 g were applied for 12 minutes. b) Continue disk-centrifuge separator, operating at 10,000 rpm and a constant feeding of 40 L/h using a peristaltic pump. This step produced supernatant 1 (S1) and pellet 1 (P1).

Neutralization step was conducted on the supernatant (S1) obtained by adding acid (3M HCl, 700 mL) until the desired pH (i.e. pH 5) is reached using the same calibrated pH-probe. Once, pH 5.0 was stable, a CMC (carboxymethyl cellulose) solution was added. This solution is prepared 24 hours in advance to facilitate hydration of the CMC and its full dispersion. This solution is prepared using a large volume orbital shaker and the CMC concentration is 1% w/v. Final solution is very viscous, but once made is stable for several days.

At this point (pH=5.0) the CMC solution is added to a final volume of 1:10 (v/v), which means, 4 L are added to a final volume of 40 L of S1 solution. After mixing the neutralised S1 solution with CMC, a second centrifugation step (same conditions than previous) is performed. The pellet (P2) is collected, and the moisture content was analysed, giving as a result an average of 75% water content.

Finally, P2 is frozen at −20° C. for at least 12 hours, (blast freezer will reduce this period of time) as a previous step for freeze drying. Frozen P2 was freeze dried under following conditions: plate temperature 35° C.; condenser temperature −50° C., vacuum 0.1 mBar, drying time 72 hours.

This process was replicated three times to ensure consistency, and the results are shown in Table 3, before and after drying. As observed, the process is very consistent and very similar results were obtained.

TABLE 3
Yield results and protein content of Process 1

| Non-dry weights of | | | Moisture |
|---|---|---|---|
| final product | Grams | | content (%) |
| Batch-1 | 4200 | | 75 |
| Batch-2 | 4030 | | 74 |
| Batch-3 | 3925 | | 75 |
| Average ± | 4051.7 ± 138.8 | | 74.7 ± 0.6 |
| standard deviation | | | |
| | | Protein | Protein |
| | | content | content |
| Freeze Dried weights | | (%) | (%) |
| of final product | Grams | 5.9 factor | 6.25 factor |
| Batch-1 | 1108 | 59.26 | 62.78 |
| Batch-2 | 1070 | 62.93 | 66.66 |
| Batch-3 | 1038 | 62.66 | 66.37 |
| Average ± | 1072 ± 35 | 61.6 ± 2.0 | 65.3 ± 2.2 |
| standard deviation | | | |

### EXAMLE 2

FIG. 2 illustrates Process 2. This process follows the same steps depicted in Process 1 to obtain S1. After this point, the differences are described. Once S1 is collected, an acidic solution (3M HCl, 900 mL in this case) is added to reach a pH value of 3.5; at this value the enzyme added has its maximum activity. Also, S1 temperature was increased to a final value of 40° C. for the same reason. Once these values have been reached, a specific volume (100 mL) of Viscozyme (>100 FBGU/g) was added and the enzymatic treatments lasted for 16 hours. After enzymatic treatment, pH was re-adjusted to a value of 5.0 using the same NaOH solution (6 M, 250 mL) and the mixture temperature was not controlled and allowed to reach room temperature. After pH was stable at 5.0 units, the same CMC solution (1% w/v) was added in the same proportion (1:10 v/v) and it was stirred for further 20 minutes.

After this point, the same steps described in Process 1 were undertaken (centrifugation, freezing, drying, milling and packaging). The moisture content of P2 after Process 2 was the same as in Process 1, with very slight variations (Table 4).

TABLE 4
Yield results and protein content of Process 2
(Involving Viscozyme)

| Non-dry weights of | | | Moisture |
|---|---|---|---|
| final product | Grams | | content (%) |
| Batch-1 | 4079 | | 75 |
| Batch-2 | 3980 | | 76 |
| Batch-3 | 4130 | | 75 |
| Average | 4063 ± 76.3 | | 75.3 ± 0.6 |
| | | Prot | Prot |
| | | content | content |
| Freeze Dried weights | | (%) | (%) |
| of final product | Grams | 5.9 factor | 6.25 factor |
| Batch-1 | 965 | 57.50 | 60.9 |
| Batch-2 | 925 | 57.73 | 61.2 |
| Batch-3 | 893 | 57.38 | 60.8 |
| Average | 9927.7 ± 36.1 | 57.5 ± 0.2 | 61.0 ± 0.2 |

### EXAMLE 3

The products using Process 1 and 2 were compared and the results are illustrated in the below Tables 5 to 12. Standard certified protocols were employed to determine the proximate composition, heavy metals and microbial load of the final products. Such analysis were conducted by an external accredited lab (Fitz Scientific, Boyne Business Park Unit 35, Drogheda, Co. Louth, A92 D52D, Ireland).

Functional tests were performed at Ashtown Teagasc laboratories following well stablished methods as reported in the scientific literature (Álvarez, C., et al., (2012). Functional properties of isolated porcine blood proteins modified by Maillard's reaction. Food Hydrocolloids, 28(2), 267-274; ÁLVAREZ, Carlos, et al. Protein recovered from meat co-products and processing streams as pork meat replacers in Irish breakfast sausages formulations. LWT, 2018, vol. 96, p. 679-685.). Such analysis provides an indication of the performance of the proteins here extracted when used as techno-functional ingredients in food formulations. Proteins extracted by means of this process show poor solubility, gelling and foaming capacity, but good to high water and oil holding capacity as well as emulsifying capacity. This is indicated for fat rich products.

TABLE 5
Nutrient composition (certified lab results)

| | Alkali + CMC + | |
|---|---|---|
| | Viscozyme | Alkali + CMC |
| Protein (%) | 60.4 | 64.9 |
| Fat (%) | 22.3 | 19.9 |
| Saturated fat (%) | 1.8 | 1.6 |
| Mono unsaturated fat (%) | 12.65 | 11.24 |
| Polyunsaturated fat (%) | 6.87 | 6.19 |
| Carbohydrates (%) | 12.1 | 11.3 |
| Dietary fibre (%) | 5.3 | 6.1 |
| Sugars (%) | 1.8 | 1.6 |
| Sodium (mg/100 g) | 928 | 451 |
| Energy (Kcal/100 g) | 480 | 472 |
| Moisture (%) | 2.1 | 1.9 |
| Ash (%) | 3.2 | 2 |
| Water activity | 0.176 | 0.141 |
| Peroxide value (meq/Kg fat) | 7 | 7.6 |

TABLE 6
Heavy metals analysis (certified)

| | | Alk + CMC + |
|---|---|---|
| | Alk + CMC | Viscozyme |
| Aluminium (mg/Kg) | <2 | 2.5 |
| Arsenic (mg/Kg) | <0.1 | <0.1 |
| Cadmium (mg/Kg) | 0.126 | 0.097 |
| Lead (mg/Kg) | <0.05 | <0.05 |
| Mercury (mg/Kg) | 0.0055 | 0.0036 |

TABLE 7
Amino acid profile obtained in the final product
and compared to original rapeseed cake.

| | Rapeseed cake | | Final extract | |
|---|---|---|---|---|
| | Average | SD | Average | SD |
| Asp | 8.52 | 0.10 | 8.07 | 0.06 |
| Glu | 23.21 | 0.32 | 19.09 | 0.07 |
| Ser | 6.70 | 0.10 | 6.75 | 0.03 |
| His | 2.08 | 1.47 | 2.99 | 0.05 |
| Gly | 7.87 | 0.15 | 9.33 | 0.26 |
| Thr | 5.35 | 0.08 | 4.37 | 0.06 |
| Arg | 7.00 | 0.13 | 7.74 | 0.05 |
| Ala | 5.73 | 0.13 | 6.07 | 0.04 |
| Tyr | 3.79 | 0.06 | 4.12 | 0.03 |
| Val | 4.68 | 0.13 | 4.76 | 0.02 |
| Met | 0.31 | 0.01 | 0.15 | 0.00 |
| Trp | 0.57 | 0.06 | 1.25 | 0.06 |
| Phe | 4.41 | 0.11 | 4.58 | 0.06 |
| Ile | 3.56 | 0.06 | 3.85 | 0.04 |
| Leu | 9.02 | 0.15 | 9.75 | 0.07 |
| Lys | 7.18 | 0.17 | 7.14 | 0.05 |
| % Essential | 37.16 | | 38.84 | |
| amino acid | | | | |

TABLE 8
Solubility

| | pH4 | pH7 |
|---|---|---|
| Solubility (%) | | |
| alk.CMC-1 | 3.95% | 6.65% |
| alk.CMC-2 | 2.45% | 6.20% |
| alk. + viscozyme + CMC-1 | 11.35% | 17.80% |
| alk. + viscozyme + CMC-2 | 13.20% | 10.95% |

TABLE 9
Colour

| | L | a | b | C* | h0 |
|---|---|---|---|---|---|
| alk. + CMC1 | 61.95 ± 0.03 | 4.01 ± 0.01 | 0.72 ± 0.04 | 4.07 | 10.12 |
| alk. + CMC2 | 61.81 ± 0.02 | 3.94 ± 0.01 | 0.43 ± 0.03 | 3.96 | 6.23 |

(Alk: Alkaline extraction)

TABLE 10
Gel Properties

| | 0.4 g/10 mL | 1 g/10 mL |
|---|---|---|
| Rapeseed protein | no gelation | no gelation |

TABLE 11
Water holding capacity (WHC) and
oil holding capacity (OHC)

| | WHC | OHC |
|---|---|---|
| | (g/100 g sample) | (g/100 g sample) |
| alk.CMC-1 | 188.00 ± 36.00 | 158.00 ± 5.65 |
| alk.CMC-2 | 188.00 ± 24.00 | 165.00 ± 4.24 |
| alk. + | 184.00 ± 0.00 | 185.00 ± 1.41 |
| viscozyme + | | |
| CMC-1 | | |
| alk. + | 162.00 ± 8.00 | 168.00 ± 5.66 |
| viscozyme + | | |
| CMC-2 | | |
| BSA standard | ND | 369.00 ± 12.73 |

TABLE 12
Phytochemicals content

| | Allyl | C22:1n9c | |
|---|---|---|---|
| | isothiocyanate | (Erucic Acid) | Phytic acid* |
| | (μg/kg) | (%) | (mg/g) |
| Raw rapeseed cake | 170 | <0.1 | 18.4 |
| alk. + CMC1 | 15 | <0.1 | <0.5 |
| alk. + CMC2 | 21 | <0.1 | n.d. |

*Analysis done internally

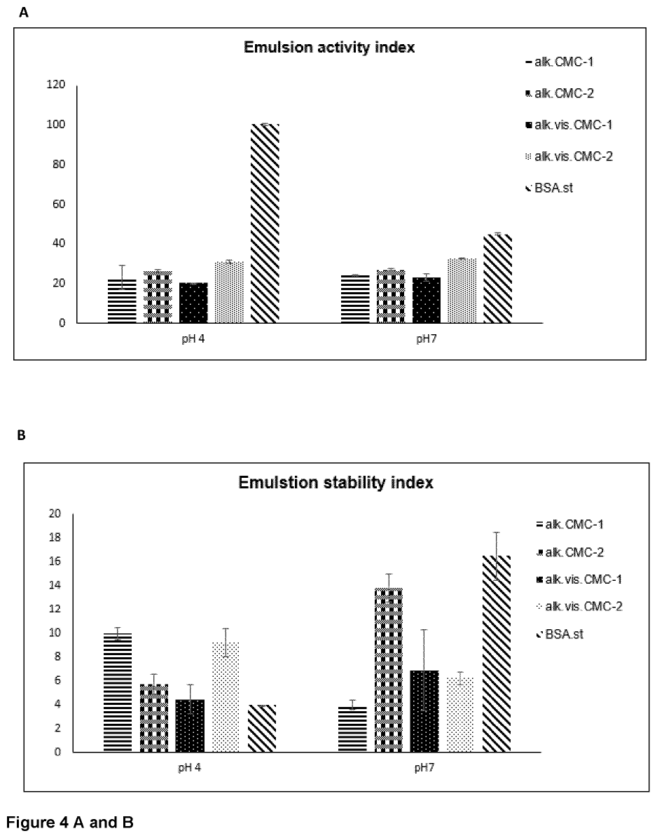

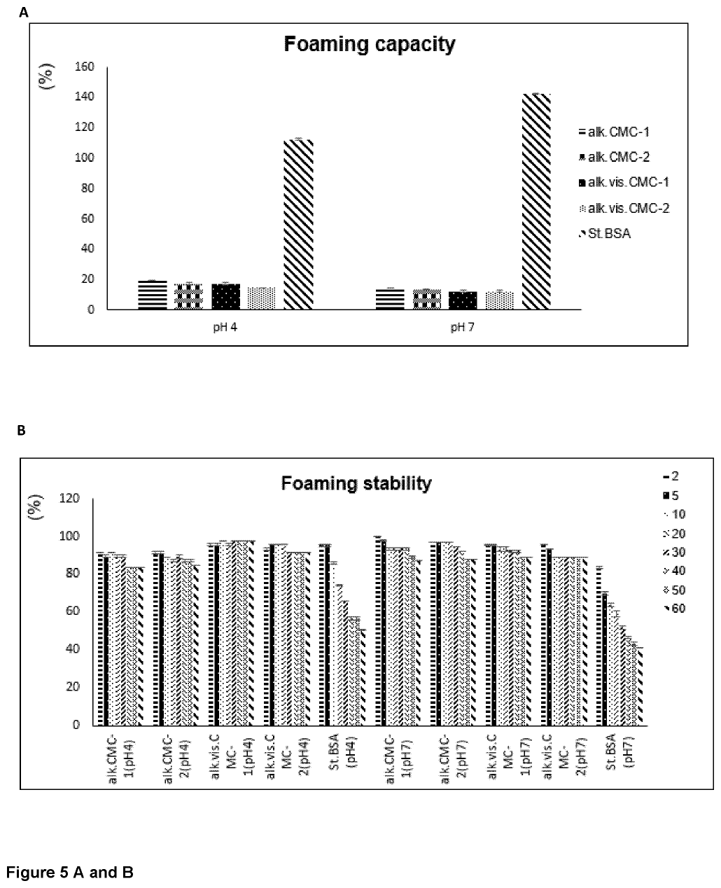

The emulsion properties are illustrated in FIGS. 4A and B. The Foaming capacity is illustrated in FIG. 5. And the foaming stability is illustrated in FIG. 5B.

### EXAMLE 4

The inventors compared the specification sheets from current products on the market with the product of the invention. This was carried out to highlight the main differences in composition and that the levels of heavy metal, phytochemicals and microbial load are within current legislation. The results are illustrated in Table 13.

TABLE 13
Comparison of the product of the invention with prior art.

| | Specification | | Alk + Vis + | Alk + |
|---|---|---|---|---|
| Parameter | Avena | | CMC | CMC |
| Protein (N × 6.25) | 33.0-43.0% | | 60.4 | 64.9 |
| Lipids | 14.0-22.0% | | 22.3 | 19.9 |
| Total | 33.0-40.0% | | 12.1 | 11.3 |
| Carbohydrates | | | | |
| Total Fibre | 33.0-43.0% | | 5.3 | 6.1 |
| Moisture | <7.0% | | 2.1 | 1.9 |
| Ash | 2.0-5.0% | | 3.2 | 2 |
| Total | <0.3 | mmol/kg | ND | ND |
| Glucosinolates | (=120 mg/kg) | | | |
| Phytate | <1.5% | | <1%* | <1%* |
| Erucic acid | | | <0.1% | <0.1% |
| Isothiocyanate/ | | | 21 | 15 |
| allyl isothiocyanate | | | | |
| (ug/g) | | | | |
| Peroxide value | ≤3.0 | mEq O2/kg | 7 | 7.6 |
| Lead | <0.2 | mg/kg | <0.05 | <0.05 |
| Arsenic (inorganic) | <0.2 | mg/kg | <0.1 | <0.1 |
| Cadmium | <0.2 | mg/kg | 0.126 | 0.097 |
| Mercury | <0.1 | mg/kg | 0.0055 | 0.0036 |
| Aluminium | <35.0 | mg/kg | <2 | 2.5 |
| Total plate count | <5 000 | CFU/g | <25000 | <200000 |
| (30° C.) | | | | |
| Enterobacteriaceae | <10 | CFU/g | <50 | <2500 |
| Salmonella sp. | Negative/25 g | | Neg | Neg |
| Yeast | | | <600 | <10000 |
| Mould | <100 | CFU/g | <100 | <100 |
| Bacillus cereus | <100 | CFU/g | <100 | <100 |
| Aerobic bacteria | ≤10 000 | CFU/g | ND | ND |
| count* | | | | |
| Total coliform | ≤10 | CFU/g | <10 | <200 |

| | | | |
|---|---|---|---|
| count* | | | |
| E. Coli* | Absent/10 g | <10 | <10 |
| Listeria | Absent | TBD | TBD |
| monocytogenes** | | | |
| Water activity** | ≤0.92 | 0.176 | 0.141 |

(Alk = alkaline (NaOH); Vis = viscozyme; CMC = carboxymethyl cellulose)

### EXAMLE 5

The inventors compared the product obtained from the method of FIG. 1 with the product obtained from the method of FIG. 6. This was carried out to analyse the glucosinolate levels and the phytic acid levels after each step of the methods. It was demonstrated that the use of higher temperature had not an impact on the glucosinolate level since they were already very low; while a reduction on the phytic acid levels of around 40% was observed when the 45° C. step was applied.

The results are provided below in Table 14 (Glucosinolate results) and Table 15 (Phytic Acid Results). Such results were obtained by using a chromatographic method (HPLC-MS/MS) for glucosinolate, and a commercial kit for Phytic acid (Megazyme, Ireland). Such methods are known in the art.

TABLE 14
Glucosinolate levels

| | | Glucosinolate Result | | Glucosinolate Result | |
|---|---|---|---|---|---|
| | | (mmol/kg) | | mmol/Kg | |
| | SAMPLE ID | Total GLS | SD | Total GLS | SD |
| 1 | Raw Canola | 9.601933 | 0.377284 | 9601.933 | 377.284 |
| 2 | Solid Fraction-Step 4-45° C. Treatment | 0.005724 | 0.000296 | 5.724 | 0.295517 |
| 3 | Solid Fraction-Step 4-25° C. Treatment | 0.005144 | 9.98E−05 | 5.144 | 0.099766 |
| 4 | Final Canola Powder-45° C. Treatment | 0.005072 | 1.53E−05 | 5.072 | 0.015315 |
| 5 | Final Canola Powder-25° C. Treatment | 0.005109 | | 5.109 | 0 |
| 6 | Final Canola Powder-Washed & Oven | 0 | 0 | 0 | 0 |
| | Dried @40° C.- 45° C. Treatment | | | | |
| 7 | Final Canola Powder-Washed & Oven | 0.005144 | 0 | 5.144 | 0 |
| | Dried @40° C.-25° C. Treatment | | | | |

TABLE 15
Phytic acid levels.

| | Phytic acid | SD |
|---|---|---|
| Sample | (g/100 g) | (g/100 g) |
| Batch 1-45° C. Treatment | 0.258 | 0.022 |
| Batch 2-25° C. Treatment | 0.449 | 0.094 |
| Oats* | 1.99 | 0.68 |

*Positive control of Oats flour supplied by Megazyme was used. The phytic acid in oats was close to what was expected (1.77 g/100 g).

1. A method of protein recovery from a plant by-product, the method comprising the steps of;
isoelectric solubilisation of the plant by-product with an alkali solution to provide a first solubilised protein fraction,
separating the first solubilised protein fraction from unsolubilised plant matter; and
flocculating the first solubilised protein fraction with an amount of a flocculating agent at a pH of between 4.5 and 5.5, to precipitate the protein.

2. The method of claim 1, wherein the flocculating agent is selected from the group consisting of:
sodium hexametaphosphate, alginate, carboxymethylcellulose (CMC), polyacrylic acid, and tannic acid.

3. The method of claim 2, wherein the flocculating agent is CMC.

4. The method of claim 1, wherein the isoelectric solubilisation is carried out at a temperature from 40° C. to 50° C.

5. The method of claim 1, wherein the isoelectric solubilization is carried out at a temperature of 45°.

6. The method of claim 1, wherein the plant is a Brassica plant.

7. The method of claim 1, wherein the plant by-product is one produced during oil production from the seed, or seeds, of the plant.

8. The method of claim 1, wherein the plant is a rapeseed plant and the plant by-product is a rapeseed press cake (RPC).

9. The method of claim 1, wherein the precipitated protein is freeze dried and/or milled.

10. The method of claim 1, wherein the method comprises a second step of isoelectric solubilisation of the unsolubilised plant matter to provide a second solubilised protein fraction and wherein the first and second solubilised protein fractions are then combined prior to addition of the flocculating agent.

11. The method of claim 1, wherein the method comprises a second step of isoelectric solubilisation of the unsolubilised plant matter to provide a second solubilised protein fraction and wherein the first and second solubilised protein fractions are then combined prior to addition of the flocculating agent and
wherein the second step of isoelectric solubilisation is with an acid solution.

12. The method of claim 10, wherein the first and second solubilised protein fractions are combined to provide a pH of 4.5 to 5.5.

13. The method of claim 1, wherein the isoelectric solubilisation step(s) is carried out under agitation.

14. The method of claim 1, wherein the method further comprises separating the precipitated protein from soluble matter.

15. The method of claim 1, wherein the method further comprises separating the precipitated protein from the soluble matter and freeze drying the separated precipitated protein.

16. A plant protein product obtained from the method of claim 1.

17. A plant protein product comprising from 60% to 70% protein content, from 1% to 10% fibre, from 20% to 25% lipids, and from 35% to 40% essential amino acids.

18. The plant protein product of claim 17, wherein the plant protein product comprises from 62% to 66% protein content, from 5% to 6% fibre, from 20% to 22% lipids, and from 37% to 39% essential amino acids.

19. (canceled)

20. The method of claim 11, wherein the first and second solubilised protein fractions are combined to provide a pH of 4.5 to 5.5.
